(19) United States
(12) Patent Application Publication
(10) Pub. No.: US 2025/0228653 A1
(43) Pub. Date: Jul. 17, 2025

(54) GASTRO-INTESTINAL IMPLANT AND ANCHORING THEREFOR

(71) Applicant: BariaTek Medical, Paris (FR)

(72) Inventors: Yonatan GRAY, Paris (FR); Christophe NAZ, Fontainebleau (FR); Youssef BIADILLAH, Maisons-Laffitte (FR)

(73) Assignee: BariaTek Medical, Paris (FR)

(21) Appl. No.: 18/841,836

(22) PCT Filed: Feb. 28, 2023
(86) PCT No.: PCT/IB2023/000088

§ 371 Date: Aug. 27, 2024

(30) Foreign Application Priority Data
Feb. 28, 2022 (CH) 000198/2022

(51) Int. Cl. A61F 2/04; A61F 2/00; A61F 2/848; A61F 5/00

(52) U.S. Cl. CPC A61F 2/04; A61F 5/0036; A61F 5/0079; A61F 2002/0081; A61F 2002/045; A61F 2002/8483; A61F 2230/0069

(57) ABSTRACT

The invention relates to an anchor (14) for an implant (20) for use in the gastro-intestinal tract. The anchor (14) comprises a tissue contact portion (16) with a bedding region (16a). The bedding region (16a) is configured to at least partly integrate into, or to promote integration into, a wall of the gastro-intestinal tract.

The present invention relates to the field of implants for insertion into the gastro-intestinal tract, and to anchors therefor. In some non-limiting aspects, the implant is a bypass sleeve for bypassing a portion of the bowel.

Various surgical techniques, and implants, have been proposed for treating obesity and diabetes. Surgical techniques include creation of gastric pockets and gastric bypasses of the stomach, duodenum and part of the jejunum. Implants such as bypass sleeves or liners have been proposed for insertion into the gastro-intestinal tract, to bypass the duodenum and optionally part of the jejunum.

Technical challenges remain for many of these techniques. For example, the endoscopic placement and anchoring of bypass sleeves remains challenging. One technique proposed is to anchor the sleeve in the vicinity of the pylorus. However, the stomach and intestine are subject to significant motion in normal bodily function. Muscular contractions of the stomach, including at the pyloric antrum, and significant bowel motility both antegrade and retrograde complicate maintaining the sleeve in position. The muscular contraction on the stomach side and retrograde motility on the bowel side can be extreme in the case of, for example, the patient vomiting. Dislodgement either towards the duodenum, or into the stomach, can necessitate medical intervention to correct the position or to retrieve the bypass sleeve. Many existing proposals are compromised by the apparently conflicting need for secure anchoring, yet with atraumatic engagement with the body tissue, and permitting sleeve removal. One suggested approach employs a sleeve device with an integral crush-resistant gastric anchor having a smooth atraumatic surface. The crush resistance prevents dislodgement out of the stomach. The surface reduces mucosal engagement and allows easy removal of the sleeve and anchor when the sleeve is no longer required or is to be replaced.

It would be desirable to address and/or mitigate one or more of the above issues.

Broadly speaking a first aspect of the invention provides an anchor for an implant (for example a sleeve) for use in the gastro-intestinal tract, the anchor comprising a tissue contact portion with a bedding region, the bedding region configured to at least partly integrate into, or to promote integration into, a wall of the gastro-intestinal tract.

The wall of the gastro-intestinal tract may be a stomach wall, optionally in the region of the antrum, but the invention may also be used in other regions of the gastro-intestinal tract, for example, the oesophagus. As explained below, the anchor may comprise or be a stent.

As used herein, the term “integrate into” means that, either immediately on implantation or within the predetermined period following implantation, the bedding region becomes a part of the wall of the tract, by tissue proliferation into and/or around the bedding region. The bedding region may be integrated into a tissue lining or mucosa of the tract wall. The predetermined period may be less than about 4 months, optionally less than about 3 months, optionally less than about 2 months, optionally less than about 1 month.

In general, the integration of the bedding region into tissue such as a wall of the gastrointestinal tract may be influenced and facilitated by a number of factors, for example:
a rough outer surface of the bedding region,
the bedding region of the anchor comprising a plurality of apertures,
the bedding region having a generally open exterior surface,
the bedding region comprising a lattice structure of struts,
the struts having a small peripheral (e.g. circumferential) width,
the struts having a small radial thickness,
the tissue contact portion comprising one or more projections (e.g. barbs or spikes).

It will be understood that the above factors individually may cause tissue integration and may be combined for e.g. faster or increased integration. No single factor is necessarily needed for tissue integration, however, any factor may be replaced by another one depending on the application.

Use of an anchor with a bedding region is a very different approach from prior art techniques of an anchor configured for only atraumatic temporary engagement with a tissue wall, without becoming bedded or embedded within the tissue wall.

By bedding into the tissue wall, the anchor can provide strong and reliable anchoring, without relying on a high crush resistance impinging on the ability of the tissue wall to move normally, and without risk of long term irritation of the wall due to repeated relative movement between the anchor and the tissue wall.

In some embodiments, the anchor and/or the tissue contact portion and/or the bedding region has a tubular shape, for engaging around the periphery of the tract. The anchor may comprise or take the form of a stent. For example, in the case of a gastric anchor for fitting at the antrum and/or near the pylorus, the anchor and/or the tissue contact portion and/or the bedding region may, at least in full-size and/or implanted state, have an outer peripheral dimension (e.g. circumference) of between about 50 mm and about 80 mm. The implant (e.g. sleeve) may optionally pass within the interior space of the tubular and/or the tubular space may allow passage therethrough of matter passing in the gastro-intestinal tract

At least the bedding region of the anchor may comprise a plurality of apertures for allowing tissue proliferation through the apertures. The anchor may have a generally open exterior surface. The bedding region may have a ratio of open-space to non-open-space of at least 2:1, optionally at least 3:1, optionally at least 4:1; optionally at least 5:1; optionally at least 10:1; optionally at least 100:1; optionally at least 200:1; optionally at least 500:1; optionally at least 1000:1.

The anchor and/or the tissue contact portion and/or the bedding region may comprise a lattice structure of struts defining apertures therebetween. The apertures may be open cells of a cellular lattice defined by the struts. The struts may optionally be coated (e.g. to protect the anchor from corrosive effects of digestive juices). Preferably the coating is very thin and does not occlude substantially the apertures between the struts.

The struts may have a peripheral (e.g. circumferential) width of between about 0.5 mm and about 2 mm, optionally about 1 mm. Additionally or alternatively, the struts may have a radial thickness of between about 0.2 mm and about 0.5 mm, optionally about 0.3 mm. Such relatively thin struts can facilitate cell proliferation around the struts to integrate the struts into the wall of the gastro-intestinal tract.

The surface of the tissue contacting portion and/or the bedding region (e.g. at least an outer surface) may optionally be non-smooth, e.g. rough. A rough surface may be provided on the struts of the anchor (e.g. stent) and/or on a covering of the struts. A rough surface can enhance frictional engagement between the anchor and the tissue wall until the bedding region has been sufficiently integrated into the wall by tissue proliferation. For example, a rough exterior surface may promote friction al engagement between the anchor and a mucosa of the tract wall. Additionally or alternatively, contacting the tissue wall with a rough surface, optionally with limited rubbing while the bedding portion beds in, may stimulate a healing response of the tissue wall, with increased tissue proliferation, thereby assisting with desired bedding into the wall.

Additionally or alternatively, the tissue contact portion may further comprise one or more projections (e.g. barbs or spikes) for penetrating into and/or piercing wall tissue. Such projections can enhance frictional engagement between the anchor and the tissue wall until the bedding region has been sufficiently integrated into the wall tissue by tissue proliferation. Optionally, one or more barbs or spikes may have an undercut profile, to help hook into the tissue wall. Projections, such as barbs or spikes, may also stimulate a healing response, as described above.

Additionally or alternatively, projections may be implemented as extensions of the anchor at at least one axial end of the anchor, preferably at both ends. Additionally or alternatively, projections may be implemented as spikes, preferably as spikes extending outward, in a radial direction, of the tissue contact. The spikes may have a length between 1 mm and 10 mm. The spikes may be extending from at least one of the struts, preferably from all of the struts of the lattice structure of struts.

Additionally or alternatively, the projections may have a shape of an at least partial hook-shape for penetrating into tissue.

At least some of the projections (e.g. barbs and/or spikes) may be of sufficient length to penetrate at least into a mucosa lining of the tract wall, for example into a gastric mucosa of a stomach wall. At least one, optionally some, of the projections (e.g. barbs and/or spikes) may be of sufficient length to penetrate into a sub-mucosal layer of the tract wall, for example, into a muscularis of a stomach wall. In some embodiments, the projections (e.g. barbs or spikes) have a length of between about 1 mm and about 3 mm, optionally between about 2 mm and about 3 mm. Additionally or alternatively, the projections may be configured as a surface structure of the struts, e.g. by an arrangement of barbs which are small relative to the strut structure.

In some embodiments, the struts have a length of between about 1 mm and about 3 mm, optionally between about 2 mm and about 3 mm.

The anchor may be made of any suitable biocompatible material, including for example metals, metal alloys, and/or polymers. Example materials include shape memory materials, for example, alloys containing at least nickel and titanium, for example, nitinol. Optionally, the anchor may be made of, or comprise, a bioresorbable material, for example, Magnesium or Poly-l-lactic acid. An anchor made of bioresorbable material can biodegrade into the tissue wall, after an expected working life of the anchor, leaving little or no lasting material embedded in the tissue wall.

The anchor may be deployable from a first, e.g. low-profile and non-deployed, configuration suitable for endoluminal delivery into the gastro-intestinal tract, to a second, e.g. expanded and deployed, configuration for engagement with the wall of the tract. The anchor may be of a self-expanding type (e.g. made of a shape-memory material), or it may be plastically expandable (for example, made of material that can be forcibly expanded). The anchor could also use a combination of both self-expansion and plastic expansion.

Additionally or alternatively to any of the above, the anchor may further comprise at least one fastener part projecting with respect to the tissue contacting portion and/or the bedding region. The fastener part may project axially, and/or radially-inwardly, with respect to the tissue contacting portion and/or bedding region. The fastener part may facilitate attachment of the implant (e.g. sleeve) to the anchor. By providing a projecting fastener part, the fastener part can remain exposed with respect to the tissue wall even once the bedding portion has been at least partly integrated into the tract wall.

Additionally or alternatively to any of the above, a second aspect of the invention provides a modular sleeve device for the gastro-intestinal tract, comprising an anchor configured to engage with wall tissue of the gastro-intestinal tract, and a sleeve extending or extendable from the anchor, the sleeve and the anchor being configured as different modules.

The sleeve may be connectable to the anchor such as to extend from the anchor.

As used herein, the term “modular” means that the sleeve and the anchor are designed such that they may be assembled together as different units to form the device, and/or designed such that they may be disassembled one from the other as different units.

In some embodiments, modular parts may be introduced to the target site in distinct stages. The anchor may be introduced and implanted in a first stage, followed by the sleeve in a second stage. The first and second stages may be carried out during the same procedure, or the first and second stages may be performed in different procedures, for example, spaced by an interval of at least 1 day, optionally at least 1 week, optionally at least 2 weeks, optionally at least three weeks, optionally at least 4 weeks.

Additionally or alternatively, whether or not the anchor and sleeve are introduced together or in distinct stages, in some embodiments, the sleeve may be separable from the anchor for removal of the sleeve, optionally leaving the anchor attached to and/or bedded in the tract wall. For example, a retrieval tool may be inserted into the gastro-intestinal tract to detach the sleeve and remove (e.g. pull) the sleeve out of the body.

The anchor and sleeve may comprise complementary fastener parts. The fastener parts may be engageable in the case of a modular device that is assembleable, and/or disengageable in the case of a modular device that is disassembleable. Example complementary fastener parts may include one or more of: male and female parts; interlocking male-male parts; interlocking female-female parts; snap-fit parts; one of more mushroom headed fastener parts; one part that hooks on to a complementary part; magnetic fastener parts; adhesive fastener parts (e.g. at least one part carrying a peelable pressure sensitive adhesive).

In some embodiments, at least the fastener part of the anchor is re-usable, allowing either the same sleeve, or a replacement sleeve, to be fastened to the existing anchor. The anchor can thus serve as a re-usable dock for sleeves, without having to remove and replace an anchor with each new sleeve placement.

Additionally or alternatively to any of the above, a third aspect of the invention provides a sleeve suitable for use with an anchor of the first aspect, and/or suitable for use in a sleeve device of the second aspect, the sleeve comprising a sleeve fastener part mateable with a complementary fastener part of an anchor.

In some embodiments, the sleeve may, in a state in which it is non-coupled to an anchor, be placed in the gastro-intestinal tract and coupled to an anchor that is already in place in the gastro-intestinal tract.

The sleeve may be absent any anchor for directly anchoring the sleeve to tissue and/or anatomy of the gastro-intestinal tract.

Additionally or alternatively to any of the above, a fourth aspect of the invention may provide an anchor for an implant (for example a sleeve) for use in the gastro-intestinal tract, the anchor comprising a tissue contact portion, optionally with a bedding region, the tissue contacting portion comprising or further comprising at least one deployable projection for penetrating tissue, the projection having a generally flat configuration when the anchor is in a first, e.g. low profile and non-deployed, configuration, and the projection bending outwardly to a projecting configuration when the anchor is in a second, e.g. deployed, configuration.

Such an arrangement can enhance engagement between the anchor and the wall of the gastro-intestinal tract, while still providing a low profile and/or flat configuration for delivery to a target site. In the flat configuration, the projection is retracted into the plane or wall of the anchor. In the deployed configuration, the projection may provide directional penetration of wall tissue, to resist migration in a specific direction. For example, the projection may resist antegrade migration of the anchor, for example, proximally.

Additionally or alternatively to any of the above, a fifth aspect of the invention provides a method of anchoring, for an implant in the gastro-intestinal tract, the method comprising:
inserting at least one delivery instrument into the gastro-intestinal tract to access a target location; and
using the at least one delivery instrument to deploy an anchor for an implant, including deploying a tissue contact portion of the anchor into contact with wall tissue of the gastro-intestinal tract, the tissue contact portion having a bedding region configured to at least partly integrate into, or to promote integration into, the wall of the gastro-intestinal tract.

The step of inserting may comprise inserting at least one delivery instrument through a natural body orifice, for example, a patient's mouth.

The method may further comprise the step(s) of allowing and/or causing the bedding region to at least partly integrate into the wall of the gastro-intestinal tract, optionally integrate substantially entirely into the wall.

The anchor may comprise an expandable stent, and the step of deploying may comprise causing expansion of the stent from a non-deployed configuration for delivery, to a deployed configuration in contact with the wall of the gastro-intestinal tract.

The method may optionally comprise using the delivery instrument to introduce an implant (e.g. a sleeve) with the anchor as a unitary device. Alternatively, the method may comprise, after the step of deploying the anchor, a step of attaching an implant (e.g. a sleeve) to the deployed anchor.

Additionally or alternatively to any of the above, a sixth aspect of the invention provides a method of performing a follow-up procedure on a gastro-intestinal tract in which an implant device (for example a sleeve device) has already been implanted, the implant device optionally according to any preceding aspect, the method comprising:
inserting at least one tool into a gastro-intestinal tract to access the implant device;
operating the at least one tool device to detach an implant component of the implant device from an anchor of the implant device, the anchor including a tissue contacting portion with a bedding region that is at least partly integrated in the wall of the gastro-intestinal tract;
using the at least one tool to remove the detached implant component from gastro-intestinal tract, leaving the anchor in place in the gastro-intestinal tract.

The implant device may be a sleeve device, and the implant component may be a sleeve of the sleeve device.

Optionally the method may further comprise inserting at least one delivery instrument into the gastro-intestinal tract to introduce a follow-up implant component, and operating the at least one instrument to attach the follow-up component to the anchor. The follow-up component may be a replacement component, for example, a replacement sleeve, although a different type of implant component from the removed component could is also envisaged.

The step of inserting may comprise inserting at least one tool and/or delivery instrument (if used), through a natural body orifice, for example, a patient's mouth.

Although the above description highlights several aspects, features and advantages of the invention, this is merely to illustrate certain ideas and principles used, and does not limit the scope of the invention. Protection is claimed for any novel feature or idea disclosed herein and/or in the drawings, whether or not emphasis has been placed thereon.

Non-limiting embodiments of the invention are now described, by way of example only, with reference to the accompanying drawings, in which:

FIG. 1 is a schematic side view of an anchor of a first embodiment;

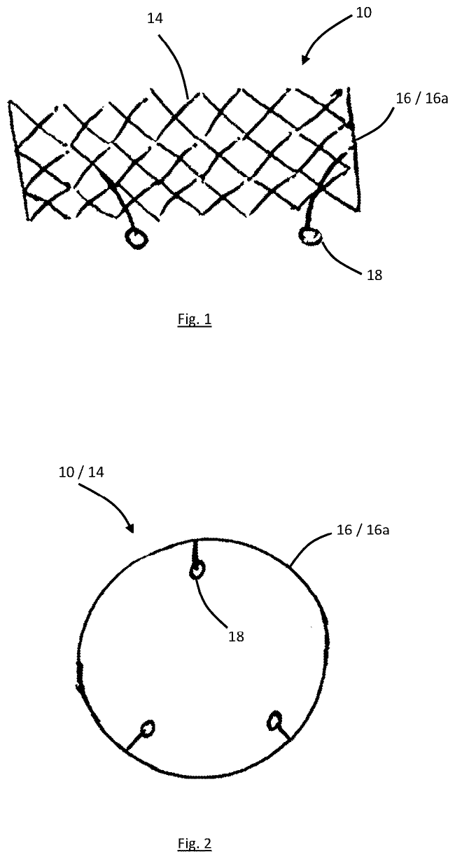

FIG. 2 is a plan view of the anchor of FIG. 1;

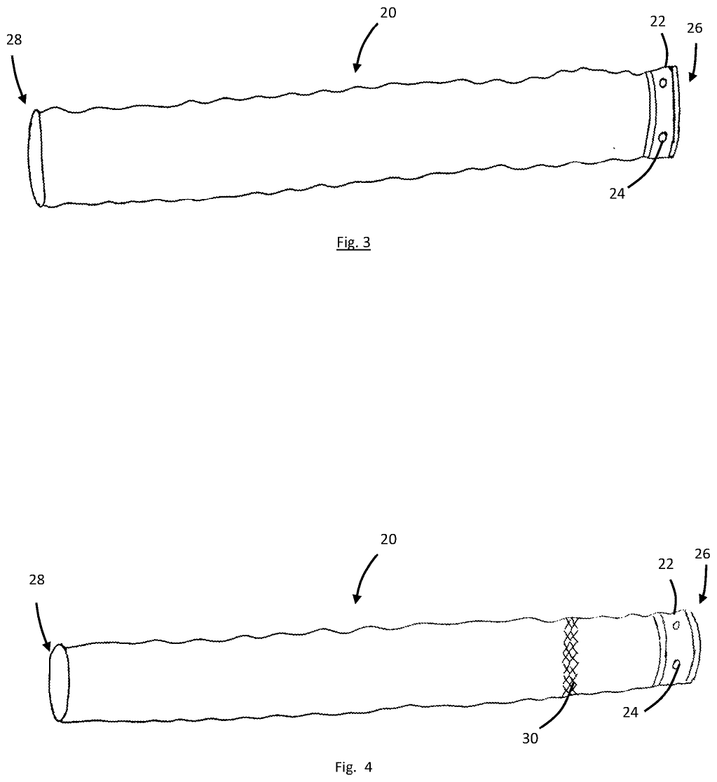

FIG. 3 is a schematic side view of a sleeve of a second embodiment usable with the anchor of the first embodiment;

FIG. 4 is a schematic side view of a sleeve of a third embodiment usable with the anchor of the first embodiment;

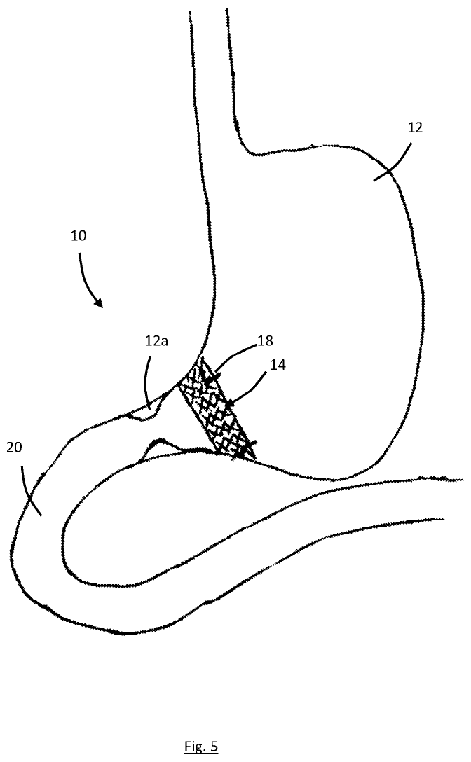

FIG. 5 is a schematic cross-section view illustrating placement of the anchor and sleeve in the gastro-intestinal tract of a patient;

FIG. 6 is a schematic side view of a further embodiment of an anchor having barbs in a deployed configuration;

FIG. 7 is an end view of the anchor of FIG. 6;

FIG. 8 is a schematic as-cut view of a cell of a modified embodiment similar to FIGS. 6 and 7; and

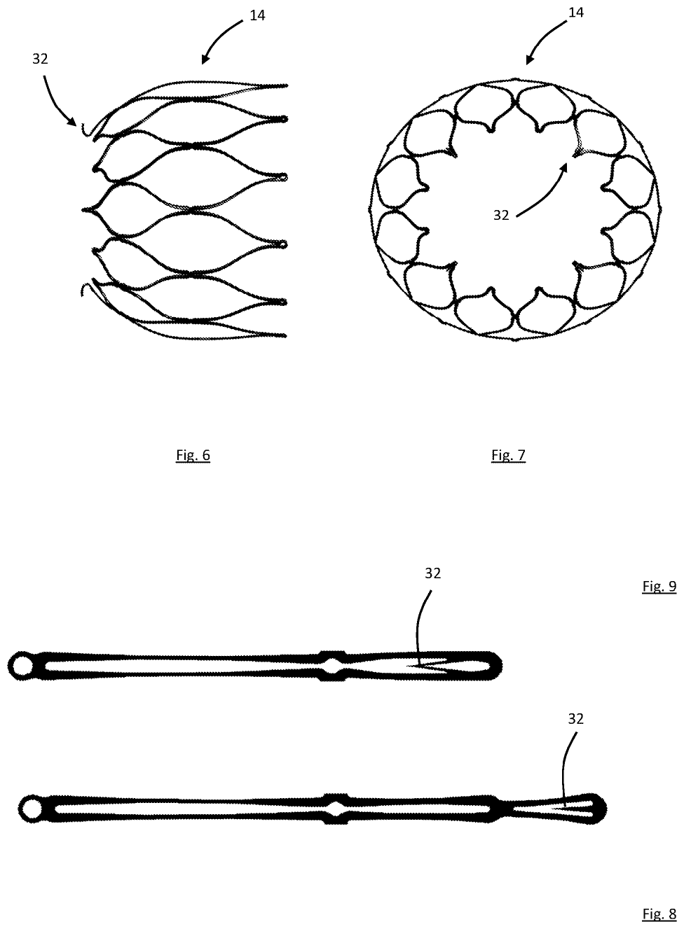

FIG. 9 is a schematic as-cut view similar to FIG. 8, showing a further modified embodiment.

Referring to the drawings, the embodiments illustrate the invention in the form of a duodenal sleeve device 10 implantable to extend from the antrum of a patient's stomach 12, through a pylorus 12a, and into the duodenum and optionally at least partly into the jejunum. However, the similar principles may be used for implanting other types of implant and/or at other locations in the gastro-intestinal tract.

Referring to FIGS. 1 and 2, the sleeve device 10 comprises an anchor 14 in the form of a stent. The anchor comprises a tubular tissue contacting portion 16 from which extend at least one, optionally a plurality, and in this example three, fastener parts 18.

The tissue contacting portion 16 includes a bedding region 16a. In this example, the bedding region 16a corresponds to substantially the entirety of the tissue contacting portion 16, but in other embodiments, the bedding region 16a could be smaller. The bedding region 16a has a lattice structure, with open apertures or cells which, in this example, have an open-mesh design. The bedding region 16a is configured to promote integration of the bedding region at least partly, preferably substantially entirely, into the wall of the gastro-intestinal tract, by tissue proliferation into the apertures and/or around the struts of the bedding region 16a.

The anchor 14 may be made of any suitable biocompatible material, including any one or more of metal(s), alloy(s) and polymer(s). The anchor struts 14 may be bare, or they may be covered with a protective coating, for example, to protect the struts from corrosive effects of digestive juices in the gastro-intestinal tract. In some embodiments, the anchor 14 may be made of a shape memory material. Additionally or alternatively, the material may be bioresorbable.

By at least partly integrating into the tract of the gastro-intestinal wall, the anchor 14 may become at least semi-permanently integrated, providing a reliable fixing for an implant, without having to rely on significant outward forces or high crush resistance for frictional and/or form fitting anchoring. If the anchor 14 is made of bioresorbable material, the anchor may biodegrade after its intended working life. Alternatively, a non-biodegradable anchor 14 could also be removed in a subsequent removal procedure, using a suitable tissue cutting or tissue grinding tool to enable the anchor to be exposed and extracted.

Although not illustrated explicitly in the first embodiment, but illustrated later, the anchor 14 may also comprise one or more projections, for example, spikes or barbs, to increase gripping and/or friction between the anchor 14 and the wall tissue of the gastro-intestinal tract until the bedding region 16a has become at least partly bedded or embedded. Additionally or alternatively, at least an outer surface of the bedding region 16a may also have a rough surface to increase frictional contact with the wall tissue.

The fastener parts 18 project axially and/or radially inwardly with respect to the tissue contacting portion 16, thereby enabling the fastener parts 18 to remain exposed even after the bedding portion 16a has become integrated in the wall of the gastro-intestinal tract. The fastener parts 18 serve as an attachment point for an implant 20.

Referring to FIGS. 3 and 4 illustrate two example embodiments of implant 20 in the form of a sleeve or sleeve component. The sleeve 20 is made of flexible polymeric material, intended to extend within at least a duodenum of a patient (e.g. see FIG. 5). The sleeve 20 has a proximal end 26 defining an entrance for placement near or at the pylorus, and a distal end 28 for placement distally within the bowel. The proximal end 26 comprises a plurality of sleeve fastener parts 24 for cooperative engagement with the fasteners parts 18 of the anchor 14. In the illustrated example, the sleeve fastener parts 24 are female parts for mating engagement with male faster parts 18 of the anchor 14, but many other type of cooperating fastener parts are envisaged, including mechanical, magnetic and adhesive fasteners. The sleeve 20 is optionally provided with a reinforcing band 22 at the proximal end 26 for distributing loads, and avoiding high stress concentrations in the sleeve material around the individual fastener parts 24.

The embodiment of FIG. 4 is similar to FIG. 3, but additionally includes a duodenal anchor 30 for fitting on the duodenal side of the pylorus. The duodenal anchor 30 can serve to prevent migration through the pylorus, and/or to provide seal contact with the duodenal wall to reduce risk of leakage of chyme around the outside of the sleeve 20. The duodenal anchor 30 may, for example, comprise a self-expanding stent mounted on or to the sleeve 20. Alternatively, the duodenal anchor 30 may comprise an inflatable cuff.

FIG. 5 illustrates placement of the implant device 10, comprising the anchor 14 and the sleeve 20, within the gastro-intestinal tract of a patient. The anchor 14 is deployed in the antrum of the stomach 12, on the stomach side of the pylorus 12a. The sleeve 20 extends distally through the pylorus into the duodenum at least.

The anchor 14 and sleeve 20 may be implanted as a combined unit, with the sleeve pre-attached to the anchor 14 by means of the cooperating fastener parts 18 and 24. Such a technique allows implantation of a complete device in a single procedure.

Alternatively, the anchor 14 may be implanted in a first stage of a procedure, and the sleeve 20 inserted in a second stage after the first stage. The first and second stages may be part of the same procedure on the patient, or they may be separated in time by, for example, at least 1 day, optionally at least 1 week, optionally at least 2 weeks, optionally at least 3 weeks, optionally at least 4 weeks.

Should it be desired to remove the sleeve 20 after a period of use, a tool may be used to access the sleeve 20 and detach the cooperating fasteners 18 and 24 to remove the sleeve 20 leaving the anchor 14 in place. The projecting fastener parts 18 can allow access even if the bedding portion 16a of the anchor is already integrated in the wall of the gastro-intestinal tract. If desired, a replacement sleeve 20, or a different implant (not shown) may be placed and attached to the existing anchor 14 to continue treating the patient.

FIGS. 6-9 illustrate examples of barbs 32 implemented as part of the stent structure of the anchor 14. In the example of FIGS. 6 and 7, barbs 32 are implemented as extensions of the stent structure at at least one axial end of the stent (optionally both ends). When the anchor 14 expands upon deployment, the barbs 32 bend through an angle of at least 90°, optionally at least 100°, optionally about 180°, to provide an at least partial hook-shape for penetrating into tissue, and opposing migration in a direction of the point of the hook shape.

FIGS. 8 and 9 illustrate as-cut views of single cells of anchor stents 14. When deployed, the anchors may have a funnel configuration similar to FIGS. 6 and 7.

In the example, of FIG. 8, barbs 32 are implemented within an interior aperture of a projecting paddle at at least one axial end of the stent (optionally both ends). In the non-deployed configuration of the anchor 14 (e.g. low profile configuration for delivery), the barbs are folded to lie generally in the plane of the paddle. Upon deployment, the barbs 32 bend outwardly, to incline away from the plane of the paddle, and penetrate into the tissue wall, to oppose migration in a direction of the point of the barb.

FIG. 9 shows a further example, similar to FIG. 8, but with the barb implemented within an interior aperture or cell of the anchor structure 14. In the non-deployed configuration of the anchor 14 (e.g. low profile configuration for delivery), the barbs are folded to lie generally within the wall of the anchor 14. Upon deployment, the barbs 32 bend outwardly, similar to the embodiment of FIG. 8.

In the illustrated embodiments, the barbs 32 are implemented at a narrow, or distal end of the anchor 14, configured for placement near the pylorus. The barbs 32 are configured to resist migration in a proximal direction. The funnel or wedge shape of the anchor (e.g. FIG. 5) provides a natural resistance to migration distally through the narrow pylorus, and the barbs provide resistance to proximal migration. If desired, barbs may also be provide at the opposite end of the anchor 14, or at any point along the length of the anchor. Barbs may optionally be configured to resist migration distally, or barbs may be provided to resist migration in either direction (e.g. individually or collectively).

It is emphasized that the foregoing description is merely illustrative of example forms of the invention, and that many modifications and equivalents can be used without departing from the scope and/or principles of the invention.

1.-22. (canceled)

23. An anchor for an implant for use in the gastro-intestinal tract, the anchor comprising a tissue contact portion with a bedding region (16a), wherein the bedding region (16a) is configured to at least partly integrate into, or to promote integration into, a wall of the gastro-intestinal tract.

24. The anchor according to claim 23, wherein the anchor and/or the tissue contact portion and/or the bedding region (16a) has a tubular shape, for engaging around the periphery of the tract.

25. The anchor according to claim 23, wherein the anchor comprises or takes the form of a stent.

26. The anchor according to claim 23, wherein at least the bedding region (16a) comprises a plurality of apertures for allowing tissue proliferation through the apertures.

27. The anchor according to claim 23, wherein the anchor and/or the tissue contact portion and/or the bedding region (16a) comprises a lattice structure.

28. The anchor according to claim 27, wherein the lattice structure is a lattice structure of struts defining apertures between the struts.

29. The anchor according to claim 28, wherein the struts have a width between 0.5 mm and 2 mm.

30. The anchor according to claim 28, wherein the struts have a radial thickness between 0.2 mm and 0.5 mm.

31. The anchor according to claim 23, wherein the tissue contact portion further comprises at least one projection for penetrating into and/or piercing wall tissue.

32. The anchor according to claim 23, wherein the anchor is deployable from a first configuration suitable for endoluminal delivery into the gastro-intestinal tract, to a second configuration for engagement with the wall of the gastro-intestinal tract.

33. The anchor according to claim 23, wherein the anchor further comprises at least one fastener part projecting with respect to the tissue contact portion and/or the bedding region (16a).

34. A modular sleeve device for the gastro-intestinal tract, comprising an anchor and a sleeve, wherein the anchor is configured to engage with wall tissue of the gastro-intestinal tract, and wherein the sleeve is connectable to the anchor such as to extend from the anchor.

35. The modular sleeve device according to claim 34, wherein the sleeve is separable from the anchor.

36. The modular sleeve device according to claim 34, wherein the anchor and the sleeve comprise complementary fastener parts (18, 24).

37. The modular sleeve device according to claim 36, wherein at least the fastener part of the anchor is re-usable.

38. A sleeve suitable for use with an anchor according to claim 23, the sleeve comprising a sleeve fastener part mateable with a complementary fastener part of an anchor.

39. An anchor for an implant, the anchor comprising a tissue contact portion, the tissue contact portion comprising or further comprising at least one deployable projection for penetrating tissue, the projection having a generally flat configuration when the anchor is in a first configuration, and the projection bending outwardly to a projecting configuration when the anchor is in a second configuration.

40. A method of anchoring an implant in the gastro-intestinal tract, the method comprising:
inserting at least one delivery instrument into the gastro-intestinal tract to access a target location, and
using the at least one delivery instrument to deploy an anchor for an implant, including deploying a tissue contact portion of the anchor into contact with wall tissue of the gastro-intestinal tract, the tissue contact portion having a bedding region (16a) configured to at least partly integrate into, or to promote integration into, the wall of the gastro-intestinal tract.

41. The method of anchoring according to claim 40, further comprise the step(s) of promoting the bedding region (16a) to at least partly integrate into the wall of the gastro-intestinal tract.

42. The method of anchoring according to claim 40, comprising the step of using the delivery instrument to introduce an implant with the anchor as a unitary device.

43. The method of anchoring according to claim 40, comprising, after the step of deploying the anchor, a step of attaching an implant to the deployed anchor.

44. A method of performing a follow-up procedure on a gastro-intestinal tract in which an implant device has already been implanted, the method comprising:
inserting at least one tool into a gastro-intestinal tract to access the implant device;
operating the at least one tool device to detach an implant component of the implant device from an anchor of the implant device, the anchor including a tissue contacting portion with a bedding region (16a) that is at least partly integrated in the wall of the gastro-intestinal tract;
using the at least one tool to remove the detached implant component from gastro-intestinal tract, leaving the anchor in place in the gastro-intestinal tract.
